(19) United States
(12) Patent Application Publication
(10) Pub. No.: US 2007/0074430 A1
(43) Pub. Date: Apr. 5, 2007

(54) ORTHOTIC DEVICE

(76) Inventor: Sven Olof Coomer, Carbondale, CO (US)

Correspondence Address: GLENN L. WEBB, P.O BOX 951, CONIFER, CO, 80433, US

(21) Appl. No.: 11/278,674

(22) Filed: Apr. 4, 2006

Related U.S. Application Data
(63) Continuation-in-part of application No. 10/605,298, filed on Sep. 20, 2003, now abandoned.

(51) Int. Cl. A43B 7/14; A61F 5/14

(52) U.S. Cl. 36/145; 36/154

(57) ABSTRACT

A dynamically molding orthotics device. The orthotics device consisting of a pre shaped counter frame containing an envelope within which a non-catalytic dynamic molding compound is sealed to interface between the pre-molded counter frame and the anatomy of the user. The counter frame of the pre molded shapes consist of irrigating canals, volume and pressure regulating pockets and relief areas for the purpose of (perpetually) dispersing and uniformly molding the compound to functionally dynamically and comfortably support and protect normal and amputated extremities and articulations of humans and animals. The molding compound is managed continuously by the pressures exerted between the continuously variable anatomical shapes of each extremity and articulation, flesh textures and bone densities, the pre molded counter frame shapes, and, the respective biomechanical dynamics of the whole body anatomy in each physical activity. These three elements combine to precisely circulate the molding compound to emphasize biomechanically sound support and comfort.

### RELATED APPLICATIONS

This application is a continuation in part of Ser. No. 10/605,298 filed on Sep. 20, 2003.

### FIELD OF THE INVENTION

The present invention relates to an improved orthotic device which is capable of providing dynamic support and protection for the feet and other parts of the body of human and nonhuman animals.

### BACKGROUND OF THE INVENTION

The feet constitute a most remarkable foundation for the human body. Academically the foot is referred to as a mobile adapter and propulsive lever. It is a portable foundation that is a most effective ambulatory, shock absorbing and propulsive system, consisting of 28 interdependent bones including the tibia and fibula to compose the essential ankle articulation.

As the foundation for the human body, the foot joint alignments and dynamic stability directly affects the alignment, posture and dynamic performance potential of the whole body. Proper alignment of the primary weight bearing joints is essential for the optimal function of the foot. The feet are designed to operate and work with irregular ground engaging surfaces. However through more recent generations the feet are exposed more and more to flat and hard surfaces. Further, they are contained within shoes that exacerbate over adaptation. It is believed that this results in excessive pronation and hyper mobility pathologies with the more common less than functionally stable feet. Most feet over adapt and others do not adapt enough. These feet constitute the majority. Normal feet appear to be in a minority.

The most commonly used method of providing protection for feet or other parts of the body such as normal or amputated extremities or for joints is to provide a cushion of resilient material underneath the extremity or around the joint. Such a cushion simply provides general padding beneath or around the relevant part and does not attempt to: provide any dynamic support or protection. As used herein, the term dynamic support and protection means support and protection which is capable of altering in response to movement of the relevant part, so that the part is supported and protected in most or all of its normal range of movements.

Underfoot orthotics have been used popularly for the past 30 years to help stabilize the pronatory motions and offer the foot a personalized and familiar surface against which to balance and perform. These orthoses are molded and fabricated according to a wide variety of theories, procedures and materials.

The state of the art until now has been to cast and mold the plantar shape of each foot in one static position, directly or indirectly, by transferring to thermally molded rigid or more elastic plastic or softer foam. The foot then has to adapt to this one static shape, regardless of activity or dynamic. Softer foams are often used to cover the specific shape to more comfortably ameliorate the shape that cannot adapt to the constantly changing shapes of the plantar aspect.

The plastics and foams unfortunately deteriorate and lose their supportive shapes. In most cases the foot is supported by a thermally molded rigid plastic or foam that represents the plantar surface of the foot in the one static molded position. However, the bones of the foot are in constant motion and every slightest change in position reflects a change in the plantar aspect shape. Therefore, with every weight bearing change, the foot dynamic is trying to adapt over this rigid singular static position shape. Therefore these supports are rarely comfortable and can create problems and injuries that are reflected in other parts of the anatomy. Select foam interfacing, or more elastic plastics are usually used to ameliorate the discomforts between the devices and the foot.

For example, U.S. Pat. No. 6,233,847 (Brown) dated 22 May 2001 discloses a footwear insole which consists of a soft cushioning foam blank to underlie the foot in order to provide general padding for the foot. A semi-rigid cap underlying the heel end of the base of the blank provides some additional support for the blank and hence for the foot also, in that region. However, this design makes no provision for the changing cushioning requirements of a foot during normal movement, e.g., walking, running.

It also is known to use a moldable foam or sheet plastics blank which can to some extent be customized to the particular foot shape of an individual user. In general, such blanks are heated to a temperature at which the foam plastics softens, and are placed in the shoe and allowed to harden while the user stands in the shoe with the foot in a predetermined position.

Another type of customized insole is disclosed in U.S. Pat. No. 5,647,147 (Coomer) dated 15 Jul. 1987. This orthotic insole incorporates an envelope lying beneath at least part of the foot. A two-part resin is injected into the envelope and then with the foot of the user positioned in place in the shoe as desired the resin is allowed to cure to provide a customized supporting surface beneath the foot.

Such molded insoles provide better support for a foot than a simple pad of cushioning foam, but have the drawback that the insoles are molded with the foot in one particular position and therefore do not offer ideal support to the foot for negotiating other positions. Thus, as the foot flexes and changes shape, as it does in every activity such as during walking, running or jumping, the foot is not correctly or adequately supported. Indeed, an insole molded to support a foot in a single position may be uncomfortable, as the foot attempts to move dynamically over and around this one predetermined shape or even tend to unbalance the person, when the foot is in a different position.

Existing orthotic systems represent the foot or other extremity in only one frozen neutral position, usually positioned in weighted or unweighted neutral subtalar joint and locked talo-navicular (mid-tarsal) joint alignment. Other molding methods tend to capture the foot shape in an already deformed and compensated position from the ideal anatomical shape that is normally typical just prior to weight bearing.

Other prior art designs seek to provide the dynamic cushioning and support by including in the insole a fluid filled cushion; the fluid may be a liquid or a gas. For example, U.S. Pat. No. 6,055,746 (Lyden) dated 2 May 2000, U.S. Pat. No. 6,158,149 (Rudy) dated 12 Dec. 2000 and U.S. Pat. No. 6,178,663 (Schoesler) dated 30 Jan. 2001 all disclose insoles of this general type.

Although a fluid filled cushion has the potential to provide effective cushioning, this design has a number of inherent problems. For example, if the cushion is very thick and the fluid compressible, it provides excellent padding but very poor stability. The user of this type of cushion is effectively trying to balance on a ball of air or liquid. However, if the cushion is thin, obviously it provides much less effective padding.

Another example of problems with the fluid filled cushions is if the fluid is virtually incompressible and the fluid envelope does not allow the fluid to move sufficiently when pressure is applied by the foot, the cushion provides very little effective or biomechanically functional padding. It follows that it is necessary for the fluid envelope to be designed so that fluid can move under applied pressure, but if the fluid is allowed to move too freely, again there is little effective padding or orthotic support for the foot and the design has poor stability, since the foot is pressing on a fluid which moves out from under the foot rapidly. Thus, it is necessary to restrict the flow of fluid from one area of the fluid envelope to another.

The above mentioned designs proposed a variety of solutions to these problems in the form of fluid flow restrictors in the cushions or seams formed in the cushions to direct flow. However, none of the prior proposals overcomes the problem of restricting or directing fluid flow within the cushion to provide an optimum level of padding without sacrificing stability. In particular, the prior proposals fail to make adequate provision for the recirculation of the cushioning fluid, so that a foot of the user does not press the cushioning fluid away from the areas of higher pressure with the first few steps, and thereafter reduce the cushioning and orthotic supporting ability of the insole because the fluid cannot return to the higher pressure areas.

Another problem that exists with typical orthotic systems is the lack of stability in the heel portion of a shoe during the heel strike. This is the point at which the foot is most vulnerable, during the weighting of the heel. Most stabilizing systems are static and uncomfortable or are ineffective.

There currently exists a problem in providing an orthotic system that will adapt to and works continuously with the most efficient dynamic and supportive needs of the foot or other extremity.

### SUMMARY OF THE INVENTION

The present invention resolves the above cited problems of existing orthotic supportive systems with a dynamically responsive orthotic support that adapts to and works continuously with the most efficient dynamic and supportive needs of the foot. The result is a complimentary suspension and energy transmission system.

A preferred embodiment of the present invention provides a prosthesis with a perpetually orthotically dynamic molding compound. The system is able to dynamically provide support to the area of the extremity at the time when the support is needed.

The system of a preferred embodiment includes a unique compound and containment method for interfacing anatomical parts to create orthotically dynamic molding prostheses and orthotics.

The containment system consists of a retaining sack that manages a newly formulated and proprietary dynamic molding compound to interface between the anatomical extremities, limbs and articulations of the human or animal anatomy and a premolded prosthetic counter frame or protective shell. The pressures and motions exerted by the activity of the anatomy continuously kneads, massages and irrigates the compound against proprietary pre molded shapes of the prosthesis or orthotics to generate optimum support, stability, control, performance and comfort at all times in every physical activity.

The compound is formulated to mold and disperse spontaneously when heated briefly in a microwave oven, or more slowly with other heat sources. The appropriate viscosity is derived according to the particular dynamic demands of each activity and the pressures exerted by the respective anatomy interfacing the prosthesis or orthotics.

The compound accumulates and then slowly discharges heat at a predetermined rate. The compound stabilizes warmth by thermal exchange with the respective anatomy as the accumulated heat slowly discharges.

Additional compound can be manually injected or withdrawn through an access port hole under the medial arch with a single barrel syringe like injector, to accommodate higher arch morphologies. To reduce the volume for lower arches the compound can be heated and massaged out through the port hole.

A preferred embodiment of the present invention provides an orthotic device for providing cushioning and support for parts of the body. The device includes a rigid or semi rigid counter frame which is molded to a predetermined shape. A flexible sack containing moldable paste is arranged to overlie at least part of said counter frame. The surface of the counter frame in contact with the sack is contoured so as to control the directions of flow of the moldable paste when pressure is applied by a part of the body to the surface of the sack opposite to the counter frame in use. A contact layer is used to overlie the sack.

The contact layer and the sack may be incorporated into a single unit, or a separate contact layer may be arranged to overlie the sack. The sack may be dimensioned to the size of the whole of the part of the body to be cushioned, or may be smaller in size.

The device of the present invention initially has been developed for use as a footwear insole, and will be described with especial reference to this application. However, it will be appreciated that the device of the present invention also may be used to support and cushion any of a number of parts of the body, e.g., normal or amputated extremities, joints, heads or backs.

It is envisaged that the device of the present invention would be suitable for use as a liner for a prosthesis, as a support for a damaged (injured) joint such as an ankle joint, as a liner for protective padding or a helmet, boot and shoe, or as a liner for a saddle for a pack or riding animal.

If the device of the present invention is used as a footwear insole, the base comprises a counter frame, which may extend the full length of the insole or approximately three-quarter length; the upper surface of the counter frame is contoured and shaped to support the foot and to direct the moldable material to flow in the desired manner. The counter frame may incorporate a heel stabilizer or may be used with or without a separate heel stabilizer. The counter frame contours are designed to deform under load in such a way that the contours direct the flow of paste between the foot and the counter frame. The heel stabilizer may be used independently of the counter-frame, in combination with a conventional insole

The flexible sack is dimensioned so as to overlie the counter frame. Preferably, the contact layer extends the full length of the insole and is formed of a soft and deformable sheet, which deforms readily under pressure by the foot of the user.

Preferably, the counter frame, flexible sack and contact layer are permanently secured together to form a single unit.

The device of the present invention may be used as a separate insertable device for insertion into, e.g., footwear or the contact surface of a prosthesis, or may be built into the article with which it is to be used.

By way of example only, a preferred embodiment of the invention in the form of a footwear insole will be described in detail with reference to the accompanying drawings.

### BRIEF DESCRIPTION OF THE DRAWINGS

FIG. 1 is an isometric view of a preferred embodiment of the invention, in the form of an insole for the left foot.

FIGS. 2-5 are diagrammatic side views showing a foot in combination with an orthotic system of the embodiment of FIG. 1 going through a typical sequence of positions which occur during normal walking.

FIG. 6 is a cross section on lines 6-6 of FIG. 1, that relates to the foot and orthotic in the position of FIG. 2 showing the orthotic insole substantially undeformed.

FIG. 7 is a cross section on lines 7-7 of FIG. 1, that relates to the foot and orthotic in the position of FIG. 3 showing the orthotic insole slightly deformed.

FIG. 8 is a cross section on lines 8-8 of FIG. 1, that relates to the foot and orthotic in the position of FIG. 3 showing the orthotic insole increasingly deformed.

FIG. 9 is a cross section on lines 9-9 of FIG. 1, that relates to the foot and orthotic in the position of FIG. 3 showing the orthotic insole increasingly deformed.

FIG. 10 is a cross section on lines 10-10 of FIG. 1, that relates to the foot and orthotic in the position of FIG. 4 showing the orthotic insole increasingly deformed.

FIG. 11 is a cross section on lines 11-11 of FIG. 1, that relates to the foot and orthotic in the position of FIG. 5 showing the orthotic insole increasingly deformed.

FIG. 12 is an isometric view of the under side of a heel stabilizer for the right foot.

FIG. 13 is a perspective view of a heel stabilizer and insole of a preferred embodiment of the present invention.

FIG. 14 is a perspective view of the heel stabilizer of FIG. 13.

FIG. 15 is a side view of the heel stabilizer of FIG. 13.

FIG. 16 is a perspective view of the heel stabilizer of FIG. 13.

FIG. 17 is a sectional view of the heel stabilizer of the embodiment of FIG. 13 unweighted.

FIG. 18 is a sectional view of the heel stabilizer of the embodiment FIG. 13 weighted.

FIG. 19 is an illustration of an alternative embodiment using a thermal transfer layer.

FIG. 20 is another view of the embodiment of FIG. 19.

FIG. 21 is a view of fluid pressure orthotic system of another alternative embodiment.

FIG. 22 is a view of an unweighted foot using the embodiment of FIG. 21.

FIG. 23 is a view of a weighted foot using the embodiment of FIG. 21.

FIG. 24 is a view of an ultra thin insole using the embodiment of FIG. 21.

FIG. 25 is a view of an ankle brace prosthesis using a dynamic molding compound.

FIG. 25 is an exposed view of the embodiment of FIG. 25.

### DETAILED DESCRIPTION OF THE PREFERRED EMBODIMENTS OF THE INVENTION

The present invention, in a preferred embodiment, provides devices and methods for providing dynamic molding of orthotic devices. A preferred embodiment of the present invention is described below. It is to be expressly understood that this descriptive embodiment is provided for explanatory purposes only, and is not meant to unduly limit the scope of the present invention as set forth in the claims. Other embodiments of the present invention are considered to be within the scope of the claimed inventions, including not only those embodiments that would be within the scope of one skilled in the art, but also as encompassed in technology developed in the future.

Underfoot orthotics are used to help describe one application of the principles though the invention is not confined to this specialty application. The same principles can be applied by the designer to create orthotically dynamic molding support in a large variety of applications in all types of prostheses. A few such applications are therapeutic and protective equipment, seats, helmets, footwear, knee and ankle pads, and saddle pads. The intention is to achieve the most refined levels of support, comfort and performance in all physical activities. The lingering warmth and thermal exchange qualities, when combined with assorted orthotically dynamic molding capacities, also has substantial therapeutic and medical applications.

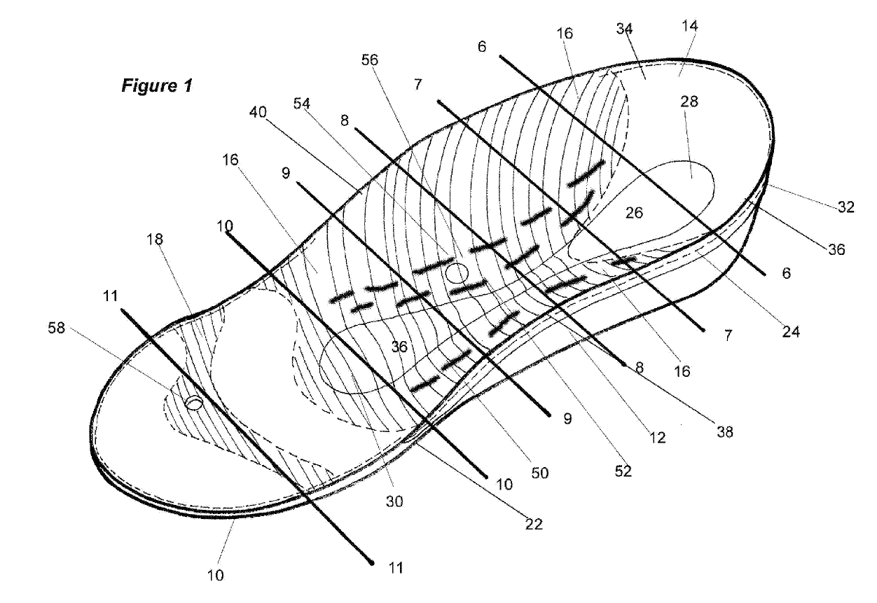

Overview of the Dynamic Orthotic Molding System A preferred embodiment of the present invention is illustrated in FIGS. 1-11 of the drawings. The dynamic orthotic molding system of this preferred embodiment includes a footwear insole 10 having a base or counter frame 12, a contact layer 14 and two separate flexible sacks 16, 18 containing a moldable paste and sandwiched between the base 12 and the contact layer 14.

In an overview of the preferred embodiment of the present invention, a dynamic molding prosthesis or orthotic is provided that constantly generates natural and locked supportive shape definitions at every moment and during every movement. The combination of the movement of the moldable paste due to the kneading action of the foot and the pre-molded counter frame shape as well as the viscosity and elasticity of the moldable paste provide the dynamic changing of the supportive shape during these movements. It is to be expressly understood that while descriptive embodiments are provided herein for explanatory purposes, the claimed inventions are not to be limited by these descriptive embodiments.

The counter frame 12 of this preferred embodiment extends the full length of the insole and forms the bottom of the insole. The counter frame 12, of this preferred embodiment, is a one-piece molding of a compression-molded foam which is elastic but substantially non-compressible material, formed with a lower surface, which corresponds in plan to the shape of the foot, and side walls 24 which extend upwards around the lower surface 22 to fit around the sides and rear most portion of the foot. This counter frame as well as alternative embodiments are discussed in greater detail below.

The lower surface 22 of the counter frame 12 is formed with a thinned or cut-out portion 26 in the shape of an elongated numeral 8. One loop 28 of the portion 26 lies underneath the area of greatest pressure applied by a heel of a user. The other loop 30 of the portion 26 lies in a position under the metatarsal or transverse arch. The thickness of the counter frame 12 over the area of the thinned portion 26 is reduced, so that the counter frame has greater flexibility in this area, and can flex downwards, (i.e., towards the ground surface underneath the insole) when pressure is applied by the foot during walking.

Flexible ribs (shown in FIG. 1 only) are formed on the lower surface 22 of the counter frame, partially surrounding the portion 26. Three such ribs 50, 52, 54 are depicted in broken lines in FIG. 1 but it must be emphasized that the ribs 50, 52, 54 are exemplary only: the number, thickness, length and positioning of the ribs can be varied widely, to suit particular requirements.

The ribs project outwards from the plane of the surface 22 and thus support the insole upon the underlying boot or shoe. In particular, the ribs suspend the portion 26, to keep the groove formed by the portion 26 open so that in use the moldable paste can move along the portion 26, as hereinafter described.

The insole can be made stiffer and more supportive by increasing the number or width of the ribs or by decreasing the spacing between the ribs. Conversely, the insole can be made softer and more flexible by using fewer ribs or by making the ribs narrower or more widely spaced.

It is envisaged that the counter frame 12 could be formed with a plurality of relatively long, closely spaced ribs, and the technician fitting the insole to a customer could trim or remove ribs as necessary to achieve the described performance characteristics for the insole.

As shown in FIGS. 1, 2, 5 and 6-11, the back wall 32 of the counter frame 12 and the adjacent rear most portions 34, 36 of the side walls 24 are comparatively high, to cup the heel of the wearer and provide stable support for the heel. As shown in the cross sectional views 7 and 8, the portion 38 of the walls 24 which extend along the inner side of the foot, i.e., adjacent the inner arch of the foot are higher than the portion 40 of the wall on the opposite side of the counter frame, to give firm and elastic support to the arch. The fact that the counter frame has raised side walls assists in directing the flow of the moldable paste by the shape and dynamics of the foot, as described in detail hereinafter. The side walls 24 gradually decrease in height towards the toe of the insole, so that the forward portion of the insole from approximately the midpoint of the foot onwards is almost flat.

The upper surface of the counter frame 12, i.e., the surface which in use is in contact with the underside of the sack 16 of moldable paste is formed with a central prominence 42 which corresponds in position to the thinned portion 26 and extends substantially the full length of the thinned portion, gradually decreasing in height from the heel to the toe of the insole. Corresponding channels 44, 46 extend along the length of the insole along each side of the prominence 42, but the channel 46 along the inner side of the insole is substantially deeper so that in use more of the moldable paste lies in this area to cushion the arch and to allow free flow of the paste. As the prominence 42 flattens towards the toe of the insole, so the channels 44, 46 also decrease in depth, as is visible from FIGS. 10 and 11.

Two separate sacks of moldable paste 16, 18 lie on top of the counter frame 12. The first sack 16 covers a major portion of the counter frame, as shown in FIG. 1. The sacks 16 and 18 may be secured in position relative to the counter frame 12 in any suitable way, e.g., by securing the perimeter of each sack to the underlying surface of the counter frame or the contact layer 14. The moldable paste may be free to move within each sack without restriction, or the sacks may be subdivided e.g., by vertical stitching to provide specific flow channels for the moldable paste, as in another preferred embodiment of the present invention. In this preferred embodiment, the sacks are not subdivided in any way. Instead it is the relationship of the foot dynamics and the base contours that control the flow of the paste. This ensures that the paste can move freely in use and can recirculate easily.

The sack 16 extends from just in front of the heel area, (the calcaneus contact point) to just short of the ball of the foot, but with a rear extension 48, 50 on each side of the thinned portion 26. The sack 16 ends just beyond the forward end of the thinned portion

The sack 18 is roughly angularly shaped and covers the area to and under the metatarsal heads, under the base of the toes. For less demanding applications where a lower level of cushioning is acceptable, a layer of cushioning foam could be substituted for sack 18.

Sack 16 and sack 18 each is fitted with an insertion valve or plugged opening 56, 58 through which additional paste can be inserted into or withdrawn from the sack, to suit the supportive requirements of a particular user.

The contact layer 14 is a flat sheet of material of the same shape in plan as the upper surface of the counter frame 12. The contact layer 14 may be made of one or more layers of any suitable material, e.g., leather, fabric, foam or plastics material and may include an additional cushioning layer.

The counter frame sacks 16 and 18 and contact layer 14 may be secured together using any suitable known techniques (e g sewing, welding, gluing). Also, the sacks 16 and 18 may be formed integrally with the contact layer. The sheet cushioning material sold under the U.S. trade mark SKYDEX may be a particularly suitable material for the contact layer 14, and it is envisaged that the material also could be used to form an additional or substitute covering layer lying between the upper surface of the counter frame 12 and the sacks 16 and 18.

In use

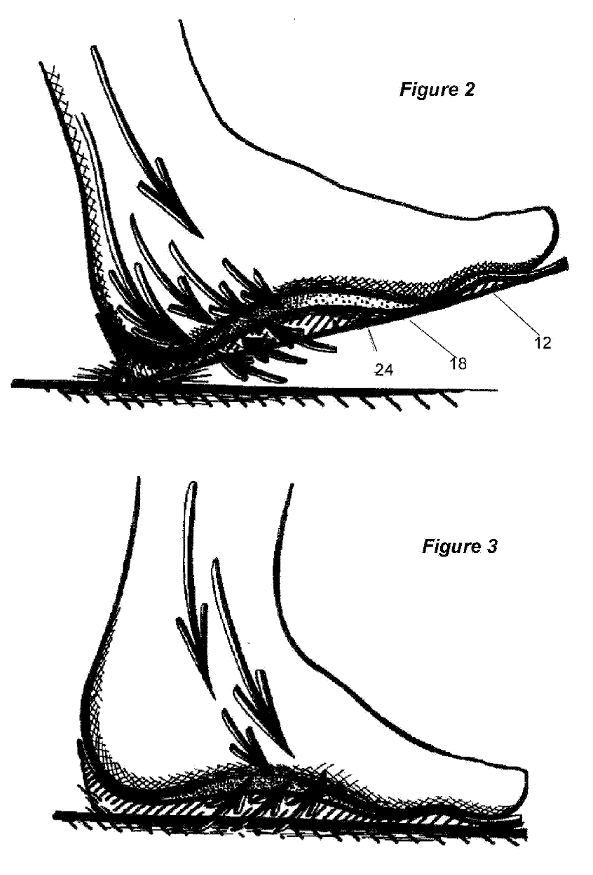

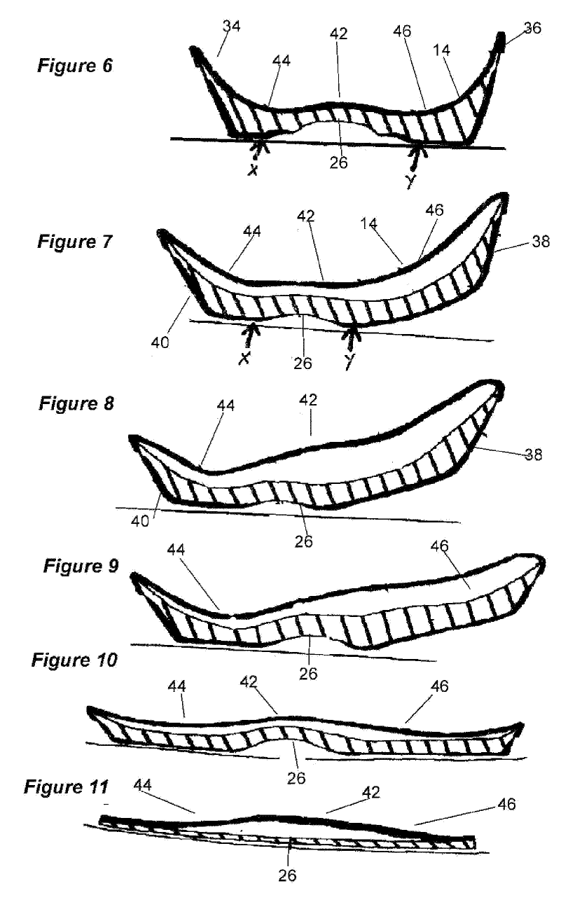

The above insole will now be described in use; the deformation which the insole undergoes throughout a normal walking gait cycle in use is shown by a comparison of FIGS. 6 with FIGS. 7-11, and the position of the foot during each stop is shown by the sequence of FIGS. 2-5. In FIGS. 2-5, the pressure exerted by the wearers weight on the insole and ground are indicated by broad headed arrows.

Referring first to FIGS. 2 and 6, at the first part of a step (heel strike), the foot is angled at an acute angle to the surface of the ground, with only the heel touching the ground, as shown in FIG. 2. At this stage, the walker is transferring all his weight to that foot; normally the foot is slightly supinated and adapts immediately to the underlying ground by pronating inwards towards the medial aspect as the weight on the foot increases.

As shown in FIG. 6 as the heel strikes, the thinned portion 26, which is directly under the center of the heel, flattens out from the position of FIG. 6 to that of FIG. 7. This reduces the height of the prominence 42 and also causes the sides 34, 36 of the counter frame immediately adjacent the heel area to move, cupping the sides of the heel and thus stabilizing the calcaneus maintaining the integrity of the shock absorbing fat pad of the heel and shape while helping to reduce slipping of the heel relative to the insole or the shoe sole.

The ribs 50, 52, 54 are not shown in FIGS. 6-11, but the arrows X and Y indicate approximately the positions of the ribs. It will be noted that there is no paste in part of this area, the sack 16 does not extend over most of the heel portion. As shown in FIG. 2, the moldable paste in the sacks 16 and 18 is substantially uncompressed at this stage.

FIG. 3 shows the next stage in the step, in which the weight bearing on the foot is complete, and the pronatory motion ends. The modification of the insole at this stage is shown by the contrast between FIGS. 7-9. A comparison of FIGS. 7-9 with the corresponding FIGS. 10-11 shows how the increase in pressure on the portions of the insole indicated by the section lines flattens the prominence 42 and starts to compress the moldable paste in the vicinity of the prominence 42. Further, the pressure exerted by the wearer curves the sides of the insole drawing the sides of the insole in towards the foot to give additional support.

The curving of the sides of the insole in this way tends to push the moldable paste from the outer edges of the insole back towards the center line of the insole; this helps to counteract the tendency of the moldable paste to escape by moving towards the outer edges of the insole due to the greater pressure of the foot in that area.

It is at this stage that the wearer needs cushioning under the midfoot arch area, since, as shown by the broad headed arrows in FIG. 3, much of the weight of the wearer is on that area of the foot. The cushioning effect is achieved by the ability of the moldable paste to move, and it will be noted that the moldable paste is encouraged to move along the length of the insole by the depression of the prominence 42; this is facilitated by the presence of the thinned area 26.

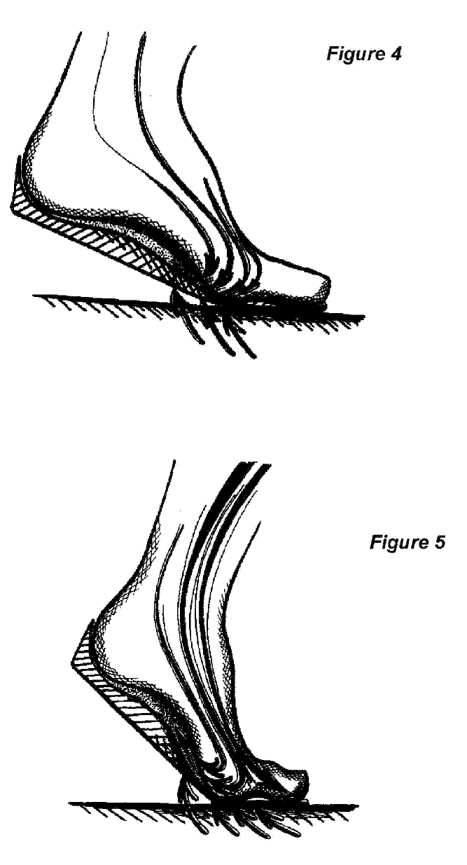

FIG. 4 illustrates the propulsive phase of the step, as the heel lifts and all the weight bearing is guided by the counter frame deformation and paste displacement to follow along the neutral axis of normal weight bearing in the gait cycle to the center of the metatarsal heads.

A comparison of FIG. 10 and 11 show how the prominence 42 is flattened in this area, which is proximal to the end of the sack 16. The wearer pressure applied adjacent the end of the sack 16 now pushes the moldable paste back towards the heel of the insole, thus reversing the direction of flow of the moldable paste which occurred during the earlier stages of the step. The fact that the sack 16 ends at about this point means that the moldable paste cannot be pushed further forward towards the toe of the insole, where it would tend to lodge permanently, thus rapidly reducing the cushioning effect of the insole.

FIG. 5 shows the final stage of the step. This is the propulsive toe off stage in which the pressure of the wearer is on the metatarsals and upon the toes, propelling the wearer forwards. At this stage, cushioning and support is required under the sulcus of the toes to activate the proprioceptive sensors and control of balance; this is supported by the sack 18. The balance of the user is positively affected by the amount of cushioning in this area, while too much cushioning in the sack 18 will not only be uncomfortable but may also indicate imbalance to the skeletal alignment of the wearers foot. As shown in FIG. 11, the sack 18 is used simply to provide a small amount of stable cushioning in this area.

The sequence of the step is now completed and this sequence is repeated by the other foot. It will be appreciated that the moldable paste not only offers cushioning without instability, but also is automatically recirculated by the wearer, so that when the wearer takes the next step, the moldable paste has returned to the initial position so that it offers continual cushioning and is neither compressed nor distorted by prolonged use. The active definition created by the propulsive toe-off happens to be the optimum shape for the plantar surface of the foot to meet again upon ground strike.

In use, the above described structure provides a unique dynamically molding of the orthotic to provide the desired support for most biomechanical movements of the foot of the user. The orthotically dynamic function of the counter supportive frame is the essential foundation needed to derive this perpetual dynamic molding function by the action of the foot anatomy to effectively massage the compound into the most desired position.

The vertical components of the soft center of pressure channels attract and irrigate the compound away from the bony prominences to comfortably fill, surround, protect and support the empty spaces underfoot. The subtleties of the continuously changing shape of the foot is captured at every moment and in every type of weight bearing.

The structure of the individual components as well as additional embodiments are discussed in greater detail below:

Counter Frame Design

As discussed above, the counter frame 12 of the above described preferred embodiment provides the support against which the moldable paste is kneaded or molded by each foot. In a preferred embodiment, the counter frame 12 is formed from EVA (etlylvinylacetate) and polyethylene foams. These foams are slit into desired thicknesses, such as 3-7 millimeters. These foams are then precut into patterns to fit into their respective mold sizes. The pieces are then heated to soften and then pressed into their respective shapes. This forms the pre-molded cradle and counter frame.

Heel Stabilizers

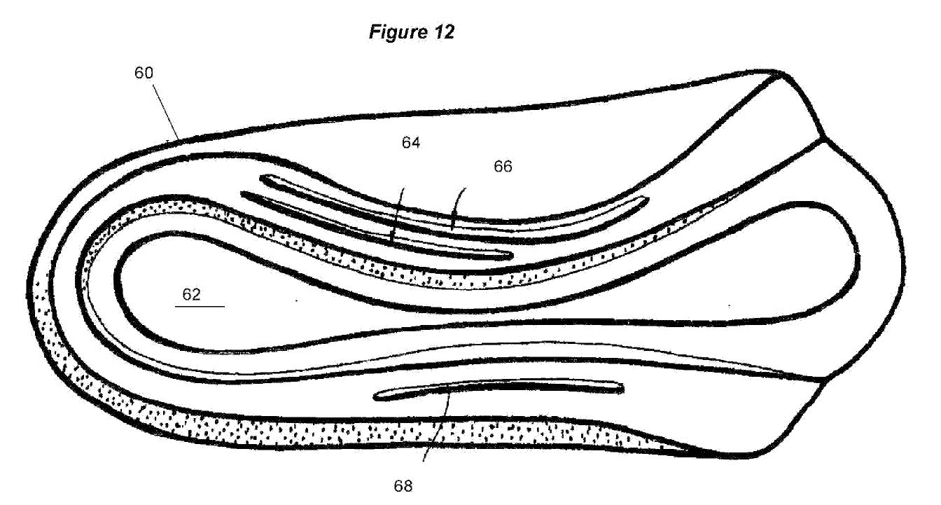

It is envisaged that for heavy duty use, the sidewalls 24 of the insole may be reinforced by inserts of a tough resilient material, e.g., composite materials. FIG. 12 shows a heel stabilizer 60 which may be used either as a reinforcement of the heel portion of the counter frame, or independently, to provide support and reinforcement for a conventional foam or pre-molded insole. This separate and interchangeable (or permanently attached) heel stabilizer enables the counter frame shape and dynamic integrity to remain independent of and unaffected by the variables in shapes, widths and supportive qualities of the various footwear.

The heel stabilizer 60 is made of a substantially rigid, hard, elastic plastic material formed to the same shape as the rear of the counter frame 12. Preferably, the heel stabilizer is fabricated in a catalyzed resin of a type which is unaffected by the constant heating and cooling of the moldable paste. Examples of such materials include such materials as nylon, polyethylene, Pebax from Dupont, or pre-assembled and pre-molded hybrid carbon fiber glass composites. Other materials may be used as well.

The base of the heel stabilizer 60 has a cut-out 62 which corresponds in shape and position to the thinned portion 26 of the counter frame 12. The underside of the heel stabilizer preferably is provided with flexible ribs 64, 66, 68 in a similar manner to the ribs 50, 52, 54 provided on the underside of the counter frame 12. The ribs 64-68 are designed and positioned, and function, in the same manner as described with reference to the ribs 50-54.

If the heel stabilizer is used in combination with a counter frame, it is simply placed over the rear portion of the counter frame and secured in position, e.g., by gluing or thermal forming. The use of the heel stabilizer in combination with the counter frame accentuates the effect of the portion 26 of the counter frame and makes the combined insole both effectively narrower and more supportive of the foot.

Stabilizer Spring

Another preferred embodiment of the present invention includes a composite or spring steel leaf spring system to provide shock absorption and control properties. This leaf spring system reinforces the contours of the counter frame to avoid permanent deformation and to add a specific dynamic response and spring return effect, and to further enhance the orthotically dynamic response behavior.

In a new and more advanced embodiment a thermally cured (pre-impregnated catalyst and adhesive) carbon-Kevlar-glass composite leaf spring system is used. This can be inserted into the molds directly under the existing foam cradle laminates, or sandwiched between the laminating foams during the compression injection molding process.

By pattern design and weave bias the designer can manage the activity, performance and endurance of the foot precisely; support, absorb and rebound the natural flexing kinematics of the foot; the specific dynamic response (rate of loading) rates required of each activity; designed according to the weight of the individual and activity demands. These dynamic precepts are possible by following the principle concept that while all feet are different, bio-mechanically they are the same and require the same needs.

These pre-impregnated temperature cured composites are formed by carefully selecting the weave bias accordingly to create the desired torsional and linear flexural dynamic properties. These patterns are designed in the shape of singular battens or from special horse shoe like patterns. The separate pre-molded battens are pre-molded in assorted stiffness and selected according to the peculiarities of each foot, the weight and performance demands of the person. These can be cemented to the underside distal aspect of the pre molded cradle, or inserted into pre embossed sleeves to ensure proper positioning.

One arm of the U can be threaded through slits in the other arm to position and stabilize the overlap, and the respective bias of the weave. The shape created is similar to an infinity symbol a sideways 8 shape. The bias of the woven composite material is selected, cut and bonded so that at the overlap their interrelationship creates a second specific structural flexural and torsional behavior to support the foot.

This pre-cut horse shoe is turned into the sideways 8 piece and is placed into the mold, under the assembled foam laminates, or between them. When the molds are closed and compressed the pre-heated temperature of the foams cures the composites in the shape pre-determined by the mold. In the case of injection or pour molding the parts are also positioned before the mold is closed and then is either filled or injected with expanding foam.

When the U wings of the horse like shoe pattern are overlapped, a tear drop shape develops inside the curve as the outside border of the curve raises simultaneously to create an intrinsic heel cup that compliments the essential deep heel cup shape of the pre-molded cradle. This overlapping method creates a heel cup without the creases, folds or distortions that develop when linear materials adapt to the deep and compound curves of the molds. The curved shapes and dynamic response properties can be created according to the bias tailoring of the woven patterns and the function evolves when the thermally sensitive material is activated with heat and cures.

The uncured soft feather spring materials are cured by the ambient heat of the foams. The horse-shoe parts or battens are assembled in the molds as rigid pre molded parts or inserted as soft materials into the molds. The parts can be inserted into the molds under the foam parts, or between the foam laminates for compression molding. In the case of pour molding or injection molding the parts would be inserted into the molds before they are closed and the foams injected.

The hole created in the base of the heel cup then acts as a Belleville Spring shock absorber. This can be supplemented by a secondary carbon compound Belleville Spring to interface the first cup hole and thereby further enhance the shock absorbing and dynamic response qualities of the pattern, as needed for different activities and personal preferences.

In this way the composite feather spring laminates system is imbedded directly into the foam molded structure, to manage the performance dynamic of the resulting device. The foams act as comfort padding and as a retainer between the cured composites and the foot of the wearer.

The previously flat U-shaped matrix cures and adopts a three dimensional curved shape of the mold to complement the external foam supportive shapes. The result is a more lively, resilient and infinitely more durable device with unique support and performance.

The tailoring of the bias of the flexible composite material can be done so that when the flat horseshoe wings are overlapped (in pretzel fashion) their opposing ends bond together and create a sideways pattern. A three dimensional tear drop shape and funnel develops inside the curve as the outside border of the curve raises to create a more functional heel cup to be imbedded into the foam laminates to complement the bubbles and center-of-pressure channel.

The deep heel cup created is an effective form of a shock absorbing Belleville spring. This further compliments the essential of the pre-molded cradle and supplements the supportive shapes of the final molded foam counter frame to result in a more lively and functionally dynamic, resilient and infinitely more durable device with unique support and performance qualities.

The bonding at the overlap of the sideways 8 weave creates a second specific reinforced laminate with structural, flexural, torsional and elasticity components available to the discretion of the designer. One important consideration is under the sustentacum tali of the calcaneus, which is in turn aligned directly under the weight bearing ankle and lower ankle joints. This allows the designer to create a predetermined dynamic response and stability for the foot to effectively flex and absorb, as if walking or running in sand, while also directing the course of weight bearing more naturally, and to manage stability at every point in the weight bearing cycle.

In this new application the dynamic forces applied by some feet transfer directly through the compound and tend to deform the structure and shape of the stabilizing counter-cradle. Therefore the cradle requires particular reinforcement to resist otherwise uncontrollable compression and deformation of the counter shapes that are essential for the irrigation of the paste within the supportive dynamic of the device.

This feather spring effectively flexes to absorb and direct the course of motions naturally, designed according to the pre-determined bias lay up of the composite glass weave, and then springs back to the original neutral position according to the desired dynamic response. The dynamic response and durability of the feather springs hybrid glass design is important in establishing long term control and stability of the desired orthotically dynamic behavior and the biomechanically sound function of the kinematic device.

Alternative Stabilizer Embodiment

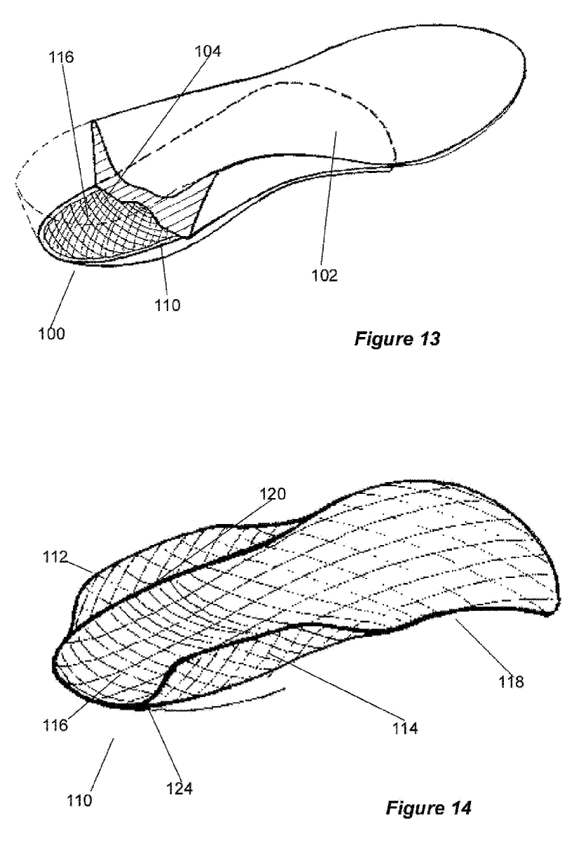

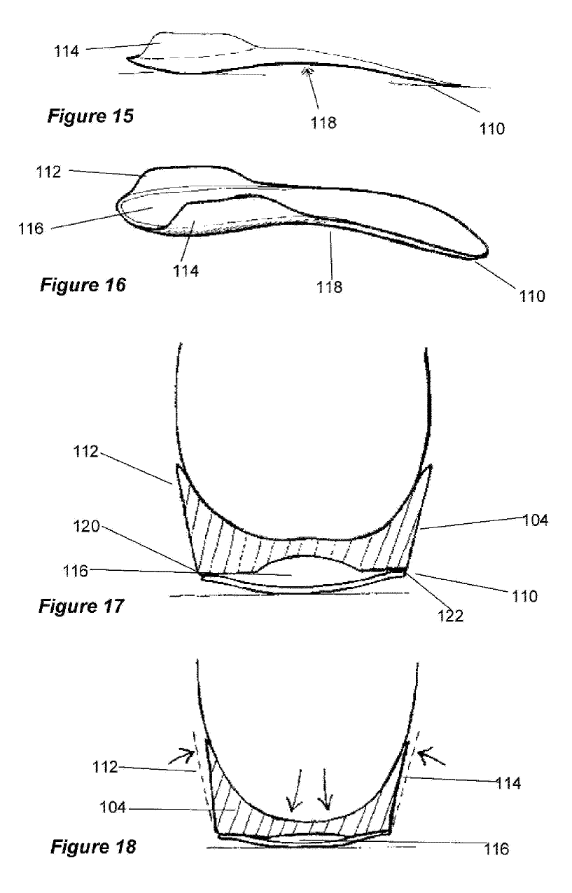

An alternative embodiment to the above described stabilizer system is illustrated in FIGS. 12-17. The stabilizer system 100 of this preferred embodiment includes a cradle or counter frame 110 beneath the contact layer or insole 102. The cradle 110, of this preferred embodiment, is formed from a tough resilient material, such as a carbon composite. The number of layers of fiberglass or other composite materials may be varied to provide the desired attributes depending on the weight and size of the user, as well as the particular foot shape and biomechanical properties. The cradle 110 includes side flanges 112, 114 extending upwardly adjacent the heel portion 104 of the insole. The cradle also includes cavity 116 that lies substantially underneath the heel portion 104 of the insole. A flexible arch bridge 118 is formed in the mid portion of the cradle 110 as well.

In use, the cradle 110 extends beneath the insole 102 so that the side walls 106, 108 of the insole are surrounded by the side flanges 112, 114 of the cradle adjacent the heel portion 104 and the cavity portion 116 of the cradle extends beneath the heel portion 104. These details are shown in FIGS. 16 and 17. FIG. 16 illustrates the unweighted heel of the user. The orthotic cradle 110 provides a slightly rounded heel base that along with the lofted arch bridge 118 allows the cradle 110 to roll and adapt as required as well as move and flex due to the various foot shapes and the dynamics from the biomechanical movement of the foot in the shoe. The insole or blank of the shoe rests on the 120, 122 of the cradle as shown in FIGS. 16 and 17.

As the heel is weighted, shown in FIG. 17, from the movement of the user, the heel portion 104 of the insole or blank is forced down against the cradle 110 as indicated by the downward arrows. The cavity 116 is flattened and the side flanges 112, 114 are pivoted inward as shown by the arrows. This also effectively decreases the width of the heel portion of the shoe thus providing additional support at this time. The greater the loading, results in an increased support and stabilization of the heel and rearfoot. This feature may be used alone or in combination with the dynamic molding features of the above described embodiments.

Vertical stabilizers may also be added around the heel portion to contain the heel tissue of the user as well as the foam material of the insole or blank of the shoe. This will further increase the stabilizing effect of the counter frame and cradle.

Moldable Compound

The moldable paste of the preferred embodiment is a high-viscosity paste with a consistency similar to that of very heavy grease, which will flow slowly even at normal operating temperatures, i.e., in close contact with the skin of the user, and therefore at a temperature typically in the range of 35 degrees C. to 40 degrees C. A paste which has too high a viscosity does not provide adequate cushioning, because it moves so slowly under foot pressure that it is virtually equivalent to a hard surface. Equally, a paste which has too low a viscosity does not provide adequate cushioning, because it simply flows away from under the foot immediately any pressure is applied.

Pastes which have proved satisfactory in use have a viscosity range which gives a flow rate of 1.5 to 7.0 grams per minute, as measured at a temperature approximately equal to body temperature, using ASTM D1238-00 Melt Flow Rates of thermoplastics by extrusion plastometer.

Preferably, the paste is non-toxic, so that there is no risk in the event of the sack accidentally being punctured. Preferably also, the paste can be heated by microwave radiation and has good heat exchange and retention properties so that it is feasible to pre-heat and soften the orthotic device to approximate skin temperatures before use. However, it is important that the paste is not too stiff when it is cold, or the insole will be uncomfortable and ineffective when used without heating because the paste will not flow properly to provide the required cushioning effect, and it will take too long for the body heat of the user to bring the paste up to the required temperature.

One suitable moldable paste constituent has been found to be a microwaveable compound sold under the trade mark by Lan Sri, (Ireviso, Italy), that is mixed with mineral or vegetable oil and a granulated cork filler to make a suitable paste for the present invention. One formulation which has been found suitable in practice is made in the following manner: an oil mixture consisting of three parts by weight vegetable oil and one part by weight mineral oil is prepared: the oil mixture is then mixed with medium quality grade/size cork particles that average about 1mm in diameter, in the proportions one part by weight oil mixture to six parts by weight cork particles. The resulting oil/cork mixture is blended in a ratio of equal parts by weight with a thermal exchange compound of the type described and claimed in the U.S. Pat. No. 5,478,988 and U.S. Pat. No. 5,494,598.

However, if it is not necessary for the moldable paste to be microwave visible, there is no requirement that the orthotic can be heated by means of microwaves, then the thermal exchange component can be omitted and the moldable paste formed simply from the oil mixture and cork particles as described above and the paste softened through the warmth of the body.

The moldable paste of a preferred embodiment includes a cork binder consisting by weight of 3 parts vegetable oil, and 1 part mineral oil. These materials are mixed, by weight, 1 part of the binder oils into 6 parts of the medium quality grade and size of cork particle. This binder and filler formulation is then blended, in a ratio of 1:1 by weight, with the thermal exchange component.

The thermal exchange component by itself has no practical application as a dynamic molding or fitting medium. It is the blending of the two components that creates the microwave visible and dynamic molding qualities in one medium.

The composite material is organic and biodegradable so there is no hazard in exposure or handling of the material. The result is a non-toxic, biodegradable, environmentally safe, recyclable composition that performs according to the most desirable spontaneous molding dynamic in a large variety of applications. Unlike two component resins there are no problems of proper mixing or time constraints of pot life and the respective chemical volatility. When properly sealed from air and evaporation, the compound can be subjected safely and effectively to an unlimited number of heating treatments for activating the molding dynamic and heat exchange properties. Likewise the compound does not have a confined shelf life time.

The performance of the functional fitting compound, when sealed in a laboratory test sample envelope, 10 cm×15 cm consists of 2 mm EVA-CORK foam, has the capacity to accumulate heat from 30 seconds in a 750 watt microwave oven. Then when set in an insulated box controlled at 0° C. to simulate winter conditions in ski boots, the core temperature of the compound begins at 120° C. and then after 5 minutes stabilizes at 90° C. for 100 minutes.

The structural and insulative properties of the EVA-Cork Foam is a proprietary formulation and quality prepared by Friuli Rubber Company, Udine, Italy. It is a base of expanded EVA foam blended with approximately 10 percent high grade granulated cork, which when prepared results in a favorably resilient texture to the hand and substantial resistance to compression set and repeated heating.

Another suitable foam backing lining for the leather liner material that interfaces the foot is neoprene rubber purchased from Spenco Corporation, Waco, Texas. Both foams have a texture that is excellent for an agreeably comfortable and soft feel that does not compression set with extended use and is unaffected by the constant heating and cooling of the dynamic molding compound.

The neoprene rubber or eva-cork is slit into 1mm sheets to minimize volume while retaining the appropriate surface texture. The neoprene foam also possesses an agreeable resistance to stretching to help stabilize the leather or other top surface materials from stretching or slipping over the compound.

The eva-cork foam or neoprene is laminated under the chosen top cover liner. This preferred embodiment uses expanded (closed cell foam) EVA (Ethyl Vinyl Acetate) laminated with polyethylene foams to create the most desirable properties in comfort, lightweight, durability and resistance to compression set. Of course other foams, particularly polyurethane and latex rubber, can be used to derive other mechanical and physical properties.

The compound may be extended under the ball of the foot and sulcus of the toes. Also the compound may be stopped behind metatarsal heads where desired by laminating the cork foam or neoprene to the counter cradle and using a soft memory foam under the sulcus to adapt to the pressures and supportive needs of the toes. The consideration may be for thinner materials under the toes in fitting into some shoes and the space consumed by the thicknesses of materials.

Softening the composite in a microwave oven is an invaluable tool for activating the fitting process, to mold to the shapes and dynamics of the foot in situ. Initial molding can be activated as instantly as taking only 10 to 12 active steps. It is important to only heat the microwave visible compound and not to affect the surrounding cradle foams that will deform with other forms of heating.

Additionally, the self-molding compound has the capacity to accumulate, stabilize temperature and exchange heat over an extended time period. When the accumulated heat wears out a thermal exchange activates between the compound and the body heat, trading and balancing temperature back and forth at around 32° C.

This ebbing and flooding of calories is physically detectable by the user.

This feature is invaluable in many everyday winter situations and for the relief of medical pathologies (such as footwear for diabetics). The devices can be heated numerous times every day before inserting them into shoes and boots.

The capacity or qualities are not diminished with repeated heating. The devices can also be deep frozen and inserted into shoes to offer cooling relief for about 30 minutes in very hot weather.

The preferred embodiment of the present invention is not limited to a singular definition of the foot in one static position, a position which may be considered academically correct. Instead the foot itself is now able to create a constantly changing variety of dynamic movements while maintaining a biomechanically stable and neutrally balanced posture depending on the dynamics of each foot and each activity. The dynamic response behavior is made possible by the essential irrigation dynamic in addition to softening by microwave heating.

The supportive shapes are constantly changing and responding directly with the plantar aspect of the foot to all aspects of gait; twisting, edging, inversion and eversion, absorption and propulsion. Without exception the orthotically dynamic molding kinetics of the device encourages an athletic behavior and response in all feet, and with many favorable physiological manifestations of improved performance, reduced fatigue and injury.

Alternative Envelopment Embodiment

In another preferred embodiment of the present invention, the sacks are subdivided into compartments, such as by vertical stitching of the two layers of the compound retaining envelope (sacks 16, 18) to allow the two materials to be separated a specified amount. It is to be expressly understood that this is a feature of one preferred embodiment and the present invention is not limited to this feature.

Vertical stitching separates and controls the space between the materials and displacement of the compound also permits the building of effective irrigation channels and static flow control pockets and release valves. At the same time this eliminates the problems of recesses, folds and lumps common with normal stitching or welding, as well as stabilizing the horizontal slipping between the two materials due to lubrication by the compound components.

In this preferred embodiment, the foot develops and concentrates the personalized shape automatically where the support is needed most and according to the particular activity. Therefore the previous problems of dynamic molding have now been solved and the most desired effect is achieved that heretofore was not possible.

In particular, this preferred embodiment provides the compound hermetically sealed within an envelope and contained within the pre molded counter frame. The pre molded counter frame topography and deformation dynamic when under pressure are designed to increase the three dimensional supportive shape according to the demands exerted by the anatomy.

The envelope or sack of this preferred embodiment may be lined with the desired impervious and friction surface textures to facilitate or hinder the rate of flow of the compound, when sandwiched between the counter frame and outer lining material. The outer lining material, such as shoe quality leather, is laminated with a material such as thin neoprene foam to control the stretching of the leather and to enhance the user friendly feeling against the foot.

Alternative Contact Layer Embodiment

In another preferred embodiment, the top surface outer lining materials create a particular surface tension due to their elasticity that affects the most desirable dynamic performance of the compound. Therefore the choice of top surface materials needs to be coordinated with the formulation of the compound.

The outer lining material and laminate is stitched and cemented around the periphery of the stabilizing frame in this preferred embodiment. The personalization port is injected with a predetermined amount of compound and then sealed with a simple 5 mm long by 4mm diameter plastic hole plug. The compound is pre-molded in the initial ready to wear shape. The device is now ready for packaging.

Heat Conductive Embodiment

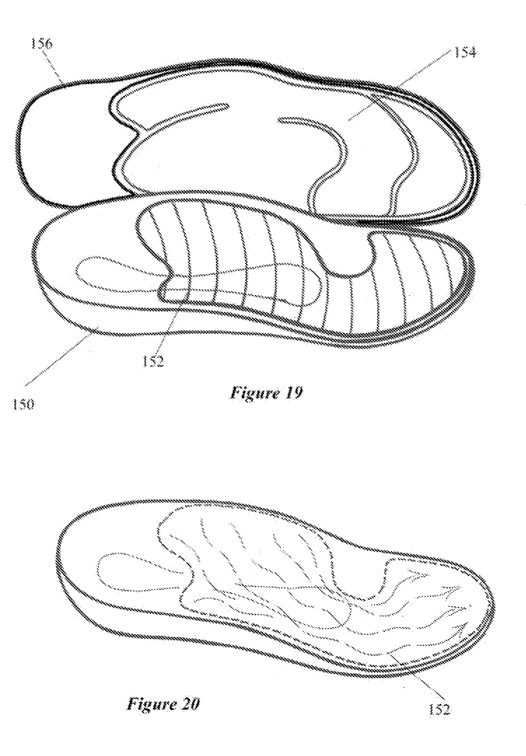

An alternative embodiment of the present invention is illustrated in FIGS. 19 and 20. This alternative embodiment uses a counter frame 150 similar to the counter frame 12 discussed above except the counter frame 150 is formed from a non-conductive (insulative) material such as but not limited to polyethylene or EVA foam. A thin self adhesive copper film 152 is attached to counter frame 150. In this preferred embodiment, the film is about 0.1 millimeter to 0.2 millimeter in thickness. It is to be expressly understood that other conductive materials could be used as well. Also the conductive film may be attached in any manner. The dynamic molding sack(s) 154 as discussed above are first attached to the top cover 160. The dynamic molding sack 154 is then attached to the counter frame over the conductive film 152.

In use, the warmth from the wearer's warm flesh at the medial arch and midfoot regions warms the molding compound in the dynamic molding sacks 154. This heat in turn is transferred onto the copper conductive film 152. The heat is transferred through the copper conductive film 152 to the front of the orthotic where it warms the toes of the wearer.

This warmth to the toes protects the vulnerable extremities that are often the first to suffer discomfort and damage from cold and wet environments. It is to be expressly understood that this feature of using a conductive film in a shoe or other device to transfer warmth to a vulnerable extremity may be provided without the molding sacks or in other types of apparel, such as gloves.

Fluid Pressure Bubble Orthotic System Embodiment

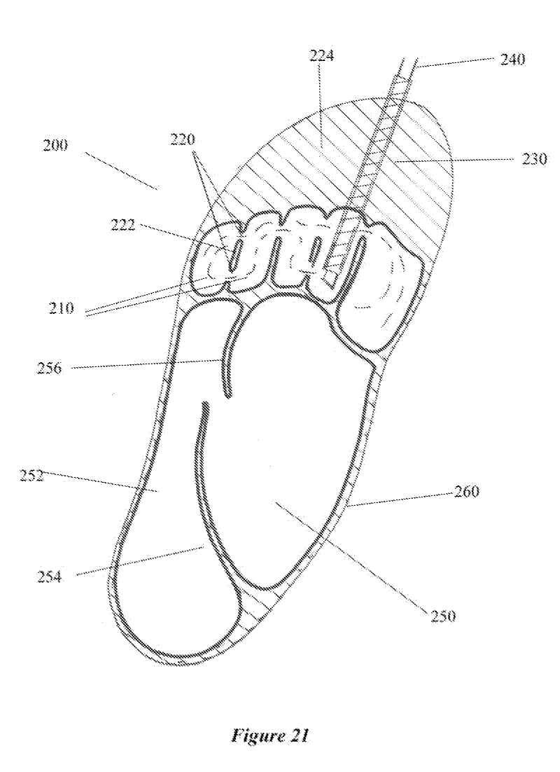

Another preferred embodiment of the present system is illustrated in FIGS. 21-23. This embodiment utilizes ambient air, gas or fluid pressure under the metatarsal heads to customize the cradling, supportive, balancing and shock absorbing action of the orthotic according to the activity and weight of the wearer. The system utilizes gas chambers or bubble chambers 210 formed between the counter frame and top cover of the insole. These chambers are formed beneath the metatarsal heads and any location that might be useful of the wearer's foot. Gates or bleeder valves 220 communicate between the chambers 210 to allow gas or fluid to circulate between the chambers. The gates 220 are designed according to the anticipated pressures and forces expected to occur in each of the chambers. The gates dissipate the gas or fluid from the pressurized chamber at a predetermined rate. These gates can be shaped to facilitate flow in one direction and to retard flow in the reversed direction. Alternatively the gates can allow flow in only one direction thus forcing the fluid or gas to exit through another gate. Also, the ability of different chambers to communicate with one another may be controlled according to the dynamics of the wearer's activity. The first metatarsal chamber may be independent of the other chambers to maintain cradling support under the joint, particular when cleated shoes are being used.

Ribs 222 may be formed or welded between the gates 220 to provide support. Also, solid section 224 may be formed or welded around the chambers for support. A soft sleeve 230 is provided in the insole to allow an inflation member 240 to be inserted therein. When the inflation member is withdrawn the sleeve collapses and seals the inflation. This provides the ability to inflate the chambers according to the wearer's activity.

An example of the use of this preferred embodiment might be for a golfer. When the golfer addresses the ball, the first metatarsal chamber empties on the medial side and inflates on the lateral side to create a stabilizing wedge under the forefoot. This helps the stance, swing and follow through of the golfer. The chambers and gates are coordinated with the selected gas or fluid medium to respond to the golfers specific dynamic needs for both walking and during the ball striking stance. As the golfer walks, the chambers return to neutral to quickly assist in a balanced walking gait.

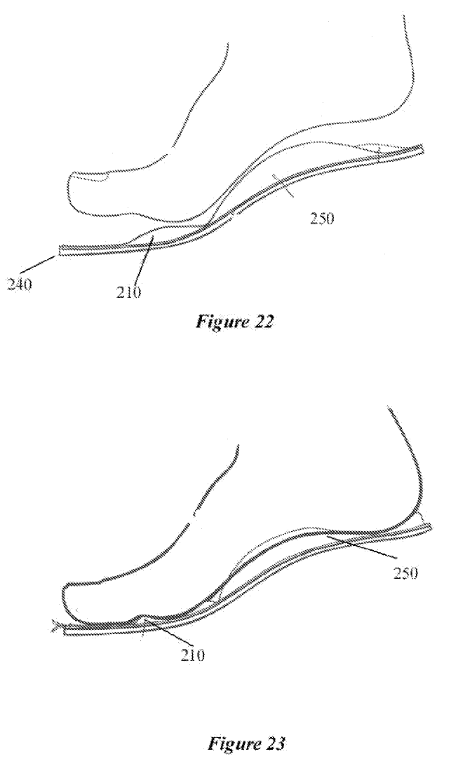

As illustrated in FIGS. 22 and 23, the ambient pressure of the ball of the foot chamber disperses weight uniformly around the ball joint when weighted while the apex of the joint just makes contact with the sole. Thus the user has shock absorbing and protective benefits without increasing or elevating the foot away from the sole. This is critical for athletes wearing cleated shoes.

This fluid pressure system can be used without the dynamic molding prosthesis system described above or in combination with as shown in FIG. 21. Molding compound chambers 250, 252 with ribs 254, 256 and gate 258 are formed in the shoe innersole as described above with the molding compound as described above as well. There may be an additional width 260 to act as a counter frame for the molding compound.

This embodiment is particularly useful for cleated athletic shoes where the cleats create pressure points against the metatarsal heads and other locations of the wearer's foot. Another critical application may be in shoes that are low volume, that is where the thickness of the innersole is to be minimized. An example of this is for lady's shoes, particularly for high heels. The high heels accentuate the pressure onto the metatarsal heads. An ultra thin shoe insert is desired for these applications.

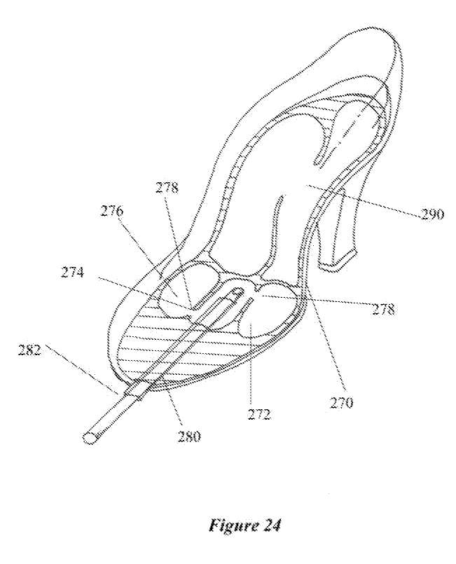

As shown in FIG. 24, shoe insert 270 includes chambers 272, 274, 276 that communicate with one another by gates 278. Sleeve 280 communicates between the ambient environment and the chambers. In this preferred embodiment, the sleeve is mounted between two welded sheets 282. An inflatable device, such as a straw is insertable into sleeve 280 to inflate the chambers. In a preferred embodiment, the sleeve 280 is self sealing to maintain the pressure in the chambers, although a valve device could be utilized as well. Also, the chambers could be inflated by a hypodermic needle device or other injection devices. The shoe insert may also include molding compound chambers 290 as well.

In use, user simply inserts a straw or other injection device through sleeve 280. The user can blow gently through straw to inflate the chambers to the proper pressure or allow gas to escape to deflate the chambers. Also, a separate inflator/deflator can be used with a pressure gauge to ensure the proper pressure.

Molding compound, as described above, can also be injected into the chambers as well. The quantity of the molding compound can be customized as desired by this same process.

Dynamic Molding Ankle Brace Prosthesis

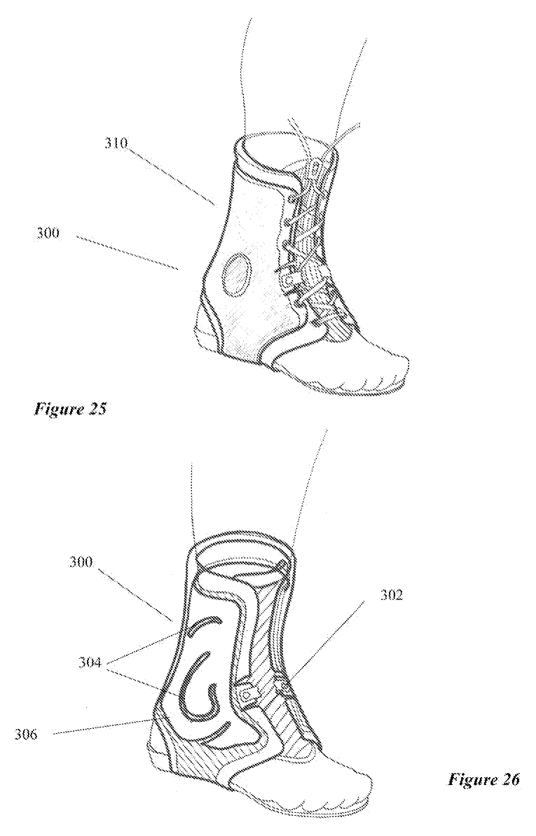

Another preferred embodiment of the present invention is illustrated in FIGS. 25 and 26. This embodiment provides an ankle brace that utilizes a dynamic prosthesis around the ankles and the dorsal aspect of the foot. This embodiment may also be used, in the proper shape for other joint bracing, such as knees, elbows, shoulders, neck, etc. The brace 300 includes an inner layer 302 that conforms to the shape of the ankle or other joint. Molding compound chambers 302 are formed in an envelope or other containment as described in the above embodiments. Ribs 304 are formed partially separating the chambers to create gates 306 for controlling the flow of the molding compound. The outer layer 310 of the brace 300 is more rigid and can be formed from reinforced and laminated webbing or other materials and combinations to create the counter frame.

The action of the molding compound under pressure from the movement of the joint protects the joints from common twisting injuries without substantially impeding the normal function and movement of the joint. The molding compound is able to move in a controlled manner through the gates as the movement of the joint occurs, but provides shock absorption and a cradling effect for unintended movements.

Alternative uses for this embodiment also include incorporating the molding chambers and counter frame into boots, including trekking, ski, snowboard, telemark and mountaineering boots. The hard outer shell of these boots may provide the dynamic counter frame and cradle system for the molding compound.

This embodiment also may be incorporated as a lining into helmets to protect from impacts without overly encumbering the wearer. This embodiment may also be used for medical purposes to protect vulnerable areas after injuries or surgeries.

These and the other descriptive embodiments were provided only for exemplary purposes and are not meant to limit the scope of the claimed inventions. Other embodiments of the present invention are considered to be within the scope of the claimed inventions, including not only those embodiments that would be within the scope of one skilled in the art, but also as encompassed in technology developed in the future.

What is claimed is:

1. A dynamically molding orthotic device, said device comprising:
a counter frame for providing support;
at least one envelopment positioned in said counter frame; and
a non-catalytic moldable material contained in said at least one envelopment for movement due to the interaction between the counter frame, the anatomy of the extremity of a user and the biomechanical movement of the extremity to provide support and protection particular to the extremity and the continuous changing stresses, shapes and supportive demands of the extremity.

2. The orthotic device of claim 1 wherein said counter frame includes:
at least one thinner portion extending under the area of greatest pressure to provide greater flexibility to allow ease of movement of said moldable material.

3. The orthotic device of claim 1 wherein said counter frame includes:
a thinner portion in shape of a numeral 8 extending under the area of greatest pressure to provide greater flexibility to allow ease of movement of said moldable material.

4. The orthotic device of claim 1 wherein said counter frame includes:
at least one rib extending along said counter frame in the area of said envelopment to assist in the movement of said moldable material.

5. The orthotic device of claim 1 wherein said counter frame includes:
multiple ribs extending along said counter frame in the area of said envelopment to form channels to assist in the movement of said moldable material.

6. The orthotic device of claim 1 wherein said counter frame includes:
a thinner portion extending under the area of greatest pressure to provide greater flexibility to allow ease of movement of said moldable material; and
at least one rib extending along said counter frame in the area of said thinner portion to assist in the movement of said moldable material.

7. The orthotic device of claim 1 wherein said counter frame includes:
side walls that extend higher at the rear portion of said counter frame to assist in the movement of the moldable material.

8. The orthotic device of claim 1 wherein said orthotic device includes:
a contact layer covering said envelopment.

9. The orthotic device of claim 1 wherein said moldable material includes:
a cork binder;
cork particle; and
a thermal exchange component

10. The orthotic device of claim 1 wherein said moldable material includes:
a cork binder formed from vegetable oil and mineral oil;
medium grade and size cork particles; and
a thermal exchange component.

11. The orthotic device of claim 1 wherein said orthotic device includes:
Stabilizer on said counter frame to provide reinforcement, said stabilizer including a tough resilient material not affected by the heating of said moldable paste.

12. The orthotic device of claim 1 wherein said orthotic device includes:
a stabilizer on said counter frame to provide reinforcement, said stabilizer including a composite material not affected by the heating of said moldable paste material.

13. The orthotic device of claim 1 wherein said orthotic device includes:
a heel portion on said counter frame;
a stabilizer on said heel portion to provide reinforcement, said stabilizer having a slightly rounded heel base shape and a lofted arch bridge to compensate for the dynamics of the shape and biomechanical movement of the extremity while providing a dynamic narrowing of the side walls of said counter frame as said heel portion is weighted to provide stabilizing to said heel portion.

14. An orthotic device for use with the support of extremities, said orthotic device comprising:
a base member having a heel portion;
a stabilizer on said heel portion to provide reinforcement, said stabilizer having a slightly rounded heel base shape and a lofted arch bridge to compensate for the dynamics of the shape and biomechanical movement of the extremity while providing a dynamic narrowing of the side walls of heel portion as said heel portion is weighted to provide stabilizing to said heel portion.

15. The orthotic device of claim 14 wherein said orthotic device includes:
said base member having a counter frame for providing support;
at least one envelopment secured in said counter frame; and
a moldable material contained in said at least one envelopment for movement due to the interaction between the counter frame, the anatomy of the extremity of a user and the biomechanical movement of the extremity to provide support and protection to the extremity.

16. The orthotic device of claim 15 wherein said counter frame includes:
at least one thinner portion extending under the area of greatest pressure to provide greater flexibility to allow ease of movement of said moldable paste material.

17. The orthotic device of claim 15 wherein said counter frame includes:
a thinner portion in shape of a numeral 8 extending under the area of greatest pressure to provide greater flexibility to allow ease of movement of said moldable paste material.

18. The orthotic device of claim 15 wherein said counter frame includes:
at least one rib extending along said counter frame in the area of said envelopment to assist in the movement of said moldable material.

19. The orthotic device of claim 15 wherein said counter frame includes:
multiple ribs extending along said counter frame in the area of said envelopment to form channels to assist in the movement of said moldable material.

20. The orthotic device of claim 15 wherein said counter frame includes:
a thinner portion extending under the area of greatest pressure to provide greater flexibility to allow ease of movement of said moldable material; and
at least one rib extending along said counter frame in the area of said thinner portion to assist in the movement of said moldable material.

21. The orthotic device of claim 15 wherein said counter frame includes:
side walls that extend higher at the rear portion of said counter frame to assist in the movement of the moldable material.

22. The orthotic device of claim 15 wherein said orthotic device includes:
a contact layer covering said envelopment.

23. The orthotic device of claim 15 wherein said moldable material includes:
a cork binder;
cork particle; and
a thermal exchange component

24. The orthotic device of claim 15 wherein said moldable material includes:
a cork binder formed from vegetable oil and mineral oil;
medium grade and size cork particles; and
a thermal exchange component.

25. The orthotic device of claim 15 wherein said orthotic device includes:
a stabilizer on said counter frame to provide reinforcement, said stabilizer including a tough resilient material not affected by the heating of said moldable paste.

26. The orthotic device of claim 15 wherein said orthotic device includes:
a stabilizer on said counter frame to provide reinforcement, said stabilizer including a composite material not affected by the heating of said moldable material.

27. A method for dynamically molding an orthotic device having a moldable material, said method comprising the steps of:
applying pressure against said moldable material by an extremity of a user during movement to allow the heat and pressure of the extremity to cause flow of the moldable material to the area of the extremity needing support.

28. The method of claim 27 wherein said method further includes the steps of:
using a heating unit to preheat said moldable material; and
cooling said heated moldable material while allowing said moldable material to retain some heat to improve the efficiency of said dynamically molding method.

29. The method of claim 27 wherein said method further includes:
providing a flexible premolded counter frame;
selecting one of a plurality of premolded carbon or plastic reinforced heel and arch stabilizers;
placing said selected reinforced heel and arch stabilizers under the device in order to adjust the shape and height of the flexible premolded counter frame to accommodate to the shape and height of each foot or joint, without the need to inject more or extract molding paste.

30. The method of claim 27 wherein said method further includes:
injecting said moldable material into or conversely extract from said device a precise amount of molding paste in order to adjust the volume necessary for properly supporting the anatomy and achieving the optimum biomechanical support and performance for the user.

31. The method of claim 27 wherein said method further includes the steps of:
providing a heel stabilizer in the frame of an orthotic system; and
causing said heel stabilizer to dynamically narrow the spacing between the side walls of said orthotic system as weight is applied against said stabilizer.

32. A thermal transfer system for footwear, said thermal transfer system comprising:
a thermal conductive layer affixed to the inner sole of the footwear for absorbing heat from the mid and rear portions of the foot of the wearer and transferring the absorbed heat to the toe portions of the foot of the wearer.

33. The thermal transfer system of claim 32 wherein said thermal conductive layer includes:
a thin conductive film.

34. The thermal transfer system of claim 32 wherein said thermal conductive layer includes:
a thin copper film.

35. The thermal transfer system of claim 32 wherein said system further includes:
an insulative barrier on the bottom surface of said thermal conductive layer.

36. The thermal transfer system of claim 32 wherein said system further includes:
a moldable conductive material;
a containment for said moldable conductive material affixed to the upper surface of said thermal conductive layer to transfer heat from the wearer's foot to said thermal conductive layer.

37. A pressurized orthotic system, said system comprising:
an insole for footwear;
at least one pressure chamber formed in said insole at a location adjacent the metatarsal heads of the wearer's foot; and
a sleeve communicating with said at least one pressure chamber to adjust the pressure in said at least one pressure chamber.

38. The system of claim 37 wherein said at least one pressure chamber includes:
at least two chambers; and
gates for communicating between said at least two chambers so that the pressure adjusts between said at least two chambers as pressure is applied from the metatarsal heads.

39. The system of claim 37 wherein said pressure chamber includes:
fluids.

40. The system of claim 37 wherein said pressure chamber includes:
gas.

41. The system of claim 37 wherein said pressure chamber includes:
moldable compound.

42. The system of claim 37 wherein said system further comprises:
a straw for insertion through said sleeve to adjust the pressure in said at least one pressure chamber.

43. The system of claim 37 wherein said system further comprises:
additional chambers formed in said insole in communication with one another; and
moldable compound in said additional chambers to flow between said chambers in accordance with pressure from the foot of the wearer.

44. A dynamic prosthesis for supporting joints, said dynamic prosthesis comprises:
an inner layer for contact against a joint of a wearer;
chambers on said inner layer in communication with one another;
moldable compound contained in said chambers; and
a substantially rigid counter frame layer on the outside of said chambers to resist the pressure of said moldable compound in said chambers as the joint moves forcing the moldable compound to flow between the chambers.

45. A dynamic helmet lining, said helmet lining comprises:
an inner layer for contact against the head of a wearer;
chambers on said inner layer in communication with one another;
moldable compound contained in said chambers; and a substantially rigid outer helmet shell on the outside of said chambers to resist the pressure of said moldable compound in said chambers as the head moves forcing the moldable compound to flow between the chambers

46. A dynamic boot lining, said boot lining comprises:
an inner layer for contact against the ankle and foot of a wearer;
chambers on said inner layer in communication with one another;
moldable compound contained in said chambers; and
a substantially rigid counter frame layer on the outside of said chambers to resist the pressure of said moldable compound in said chambers as the foot and ankle moves forcing the moldable compound to flow between the chambers.
